(19) United States
(12) Patent Application Publication
(10) Pub. No.: US 2013/0053656 A1
(43) Pub. Date: Feb. 28, 2013

(54) PHYSIOLOGICAL AND NEUROBEHAVIORAL STATUS MONITORING

(71) Applicants: Daniel J. Mollicone, Philadelphia, PA (US); Christopher G. Mott, Seattle, WA (US)

(72) Inventors: Daniel J. Mollicone, Philadelphia, PA (US); Christopher G. Mott, Seattle, WA (US)

(73) Assignee: Pulsar Informatics, Inc., Philadelphia, PA (US)

(21) Appl. No.: 13/598,398

(22) Filed: Aug. 29, 2012

Related U.S. Application Data
(60) Provisional application No. 61/528,359, filed on Aug. 29, 2011.

(51) Int. Cl. A61B 5/0205; A61B 5/01; A61B 5/0476; A61B 5/0402; A61B 5/11; A61B 5/00

(52) U.S. Cl. 600/301; 600/595

(57) ABSTRACT

A system and methods of use are disclosed for monitoring the neurobehavioral and physiological status of one or more individuals across a distributed network, the system comprising, at least in part, and according to alternative embodiments, i) a physiological sensor capable of measuring patient movement; ii) additional physiological sensors; iii) a wireless controller for monitoring polling cycles and power consumption ratings; iv) an administrative user interface for executing various executive control functions; and v) a patient interface capable of receiving input, providing output, and, optionally, administering one or more neurobehavioral tests.

### RELATED APPLICATIONS

This application claims benefit of the priority of U.S. application No. 61/528,359, filed Aug. 29, 2011.

### TECHNICAL FIELD

The presently disclosed invention relates generally to networked systems of devices and computing platforms for monitoring the neurobehavioral and physiological status of individuals. Particular embodiments monitor sleep and activity of individuals using actigraphy sensors, conduct cognitive tests, automatically transmit data to a central data store, and provide remote access by administrators.

### BACKGROUND

Monitoring neurobehavioral status of individuals may involve direct measurement using neurobehavioral test batteries, and may also involve measurement of additional physiological factors that affect performance such as sleep history. There is a general desire for methods and systems to enable such monitoring with individuals located in distributed locations outside direct supervision of clinicians or other support personnel. Quality of data collected and compliance of individuals to testing procedures may be increased by monitoring systems that streamline administrative tasks such as data transfers, and allow remote observation and quality control by automated or manual reviewers.

### SUMMARY

Particular embodiments of the presently disclosed invention provide for physiological and neurobehavioral status monitoring of individuals, including (but not limited to) medical patients. One particular aspect of the presently disclosed invention provides a system for monitoring the physiological and neurobehavioral status of a human testing subject, the system comprising: at least one physiological sensor, the physiological sensor being capable of monitoring a physiological parameter of a human testing subject; and at least one neurobehavioral testing unit, the neurobehavioral testing unit being capable of measuring the neurobehavioral status of the human testing subject.

Another particular aspect of the presently disclosed invention provides a device for monitoring the physiological and neurobehavioral status of a human testing subject, the device comprising: at least one physiological sensor, the physiological sensor being capable of monitoring a physiological parameter of a human testing subject; and a neurobehavioral testing unit, the neurobehavioral testing unit being capable of measuring the neurobehavioral status of the human testing subject; wherein the at least one physiological sensors and the neurobehavioral testing unit form an integral unit.

Another particular aspect of the presently disclosed invention provides a method, using a computer, for monitoring the physiological and neurobehavioral status of a human testing subject, the method comprising: receiving, at a computer, a physiological parameter of a human testing subject, the physiological parameter being indicative of a physiological status of the human testing subject; determining, with the computer, a comparison of the physiological parameter to a threshold; and optionally measuring, with the computer, a neurobehavioral status of the human testing subject based at least in part upon the determined comparison of the physiological parameter to the threshold.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a component-level block diagram for a system to monitor the physiological and neurobehavioral status of one or more individuals, in accordance with particular embodiments;

FIG. 2 is an illustration of a non-limiting exemplary device used to monitor the physiological and neurobehavioral status of one or more individuals, in accordance with particular embodiments;

FIG. 3 is a flowchart illustrating a method used to monitor the physiological and neurobehavioral status of one or more individuals, in accordance with particular embodiments; and

FIG. 4 is a plot showing the variation of the homeostatic process of a typical subject over the transitions between being asleep and awake, in accordance with particular embodiments.

### DETAILED DESCRIPTION

Throughout the following discussion, specific details are set forth in order to provide a more thorough understanding of the disclosed invention. The invention, however, may be practiced without these particulars. In other instances, well-known elements have not been shown or described in detail to avoid unnecessarily obscuring the invention. Accordingly, the specification and drawings are to be regarded in an illustrative capacity, rather than in a restrictive sense.

Background to Physiological Monitoring Systems

The monitoring of a human subject's physiological status is a critical function in many healthcare settings, and the prior-art contains an abundance of systems, techniques, methods, and devices for filling this need. The physiological parameters monitored by such technology may be of several varieties—including (but not limited to) electrocardiogram, electrocardiogram with respiration, invasive pressures, temperature, blood pressure, non-invasive blood pressure, blood oxygen saturation level (SₚO₂), plethysmography, carbon dioxide level (CO₂), cardiac output, transcutaneous blood gas monitoring (TcGas), and/or the like. The following patents and patent documents illustrate the use of such physiological monitoring technologies, with a focus on the network architecture of the distributed systems used therein.

U.S. Pat. No. 5,579,775 issued to Dempsey et al. on 3 Dec. 1996, entitled “Dynamic control of a Patient Monitoring System,” discloses a flexible patient monitoring system that interfaces a telemetry subsystem with a network by way of a telemetry transmission receiving system and a network controller. The telemetry subsystem includes one or more monitoring instruments (also called “physiological sensors”), a multiport transmitter, telemetry subsystem interface, a backchannel receiver module, and a telemetry docketing station. The flexible patient monitoring system may be operated to implement network-based, system-originated control of one or more of the patient monitoring functions performed in the system. A particular function that is subject to dynamic control is the transmission of the telemetry signal. (See also EPO Patent Publication No. EP0710465.) The entirety of the U.S. Pat. No. 5,579,775 is hereby incorporated herein by reference.

U.S. Pat. No. 5,752,917 issued to Fuchs on 19 May 1998, entitled “Network Connectivity for a Portable Patient Monitor,” discloses a monitor system for acquiring medical data from a patient that is distributed over at least two geographically separate patient monitoring areas and interconnected via communication network. A portable monitor (102) coupled to a patient receives and processes patient data signals from a sensor coupled to the patient. At least two patient monitor docking stations (111) are provided, each one of which is selectively coupled to the portable monitor and connected for transmission of patient data received from the portable patient monitor to the communication network. Each docking station comprises a coupler for detachably coupling the portable monitor to the docking station, a signal transfer device (108) for transferring patient-related data signals between the portable monitor and the docking station when the portable monitor is coupled to the docking station, and a signal processor for monitoring the patient-related data signals provided by the signal transfer device for developing a connection information signal when the portable monitor is coupled to the docking station, the connection information signal being applied for developing an alarm in the event that the signal processor monitors that the portable monitor is not operating correctly. The entirety of the U.S. Pat. No. 5,752,917 is hereby incorporated herein by reference.

U.S. Pat. No. 6,553,262 issued to Lang et al. on 22 Apr. 2003, entitled “Arrangement for Patient Monitoring,” discloses an arrangement for patient monitoring that includes at least one body sensor to detect a physiological parameter as body signal data and at least one of a body signal processing unit connected to the body sensor for processing body signal data, and a therapy device designed to act on the patient. In addition, the arrangement includes a monitoring center having a signal field strength evaluation means, a mobile phone terminal designed to transmit the data from at least one of the body signal processing unit and the therapy device to the monitoring center and from the monitoring center to the therapy device, and a cellular mobile phone network. The mobile phone terminal is operable in the cellular mobile phone network. The arrangement further includes a base station coordinate storage unit for storage of coordinate data and a mobile phone exchange linked to the base station coordinate storage unit for the reception of position data of the mobile phone terminal. The signal field strength evaluation means is utilized for evaluating signal field strength data reflecting the distance of the mobile phone terminal from at least one base station together with coordinate data of at least one active base station of the mobile phone network in order to determine the whereabouts of the patient. The entirety of the U.S. Pat. No. 6,553,262 is hereby incorporated herein by reference.

U.S. Pat. No. 6,659,947 issued to Carter et al. on 9 Dec. 2003, entitled “Wireless LAN Architecture for Integrated Time-Critical and Non-Time-Critical Services within Medical Facilities,” discloses a wireless local area network (WLAN) system comprising multiple access points that are distributed throughout a medical facility to provide wireless access to a hardwired network. The access points implement multiple WLAN protocols, including a real-time protocol for real-time patient monitoring (telemetry) and a standard WLAN protocol (such as IEEE 802.11 within an ISM band) for providing general-purpose wireless access. Some or all of the access points preferably implement both WLAN protocols such that the different WLANs and wireless device types share network access resources. Some or all of the access points may also include RF location-tracking modules which may be used to track locations of patients, hospital personnel, capital equipment, and/or disposable medical supplies. Also disclosed are an antenna design which may be used with the access points to improve reception (particularly for patient monitoring), and a TDMA timeslot rotation method for avoiding lockstep interference between access points that operate on the same channel. The entirety of the U.S. Pat. No. 6,659,947 is hereby incorporated herein by reference.

U.S. Pat. No. 6,749,566 issued to Russ on 15 Jun. 2004, entitled “Patient Monitoring Area Network,” discloses a system that allows patient monitoring data obtained by patient connected devices to be transferred by wireless signals to another device such as a patient monitoring processor. The same patient connected devices are used to transfer data to the patient monitor processor or a central station depending on the location of the patient. A single device is used for both a personal area network and a telemetry/transport application. The same wireless technology is used in both situations and eliminates the need to deploy more than one antenna/receiver system. Existing wireless transfer protocols such as Bluetooth are used, thereby reducing transmission power when the two communicating devices are in close proximity. The entirety of the U.S. Pat. No. 6,749,566 is hereby incorporated herein by reference.

U.S. Pat. No. 7,256,708 issued to Rosenfeld et al. on 14 Aug. 2007, entitled “Telecommunications Network for Remote Patient Monitoring,” discloses a communications network for providing continuous patient monitoring to provide critical care services from a remote location. A plurality of patient monitoring stations with associated patient monitoring instrumentation is connected over a communications network to a command center to which data flows continuously for analysis. A standardized series of guideline algorithms for treating a variety of critical care conditions are prompted to provide critical care by caregivers who monitor the progress of individual patients at remote patient monitoring stations. A smart alert system that can be flexibly set from the command center provides for patient-specific rules to be established to alert the caregivers to potential patient problems so that intervention can occur in a timely fashion. A data storage/data warehouse function analyzes individual patient information from a plurality of command centers and provides updated algorithms and critical care support to the remote command centers. The entirety of the U.S. Pat. No. 7,256,708 is hereby incorporated herein by reference.

U.S. Pat. No. 7,448,996 issued to Khanuja et al. on 11 Nov. 2008, entitled “Method and Apparatus for Remotely Monitoring the Condition of a Patient,” discloses a patient monitoring system that provides enhanced functional capability relative to known systems and provides a wireless communication link between a patient monitoring device, worn by a patient, and a local hub. The patient monitoring system is adapted to monitor various patient physiological characteristics, such as blood pressure, pulse rate, blood glucose, weight, pulse oximetry and others. The data from the patient monitoring device is wirelessly transmitted to a local hub, which, in turn, is configured to automatically transfer the data to a remote server, for example, over a public or private communications network. In one embodiment of the invention, the server is configured as a web portal to selectively, allow access to such patient physiological data by designated third parties, such as physicians, clinicians, relatives and the patient themselves. (See also PCT International Publication No. WO 03/088830.) The entirety of the U.S. Pat. No. 7,448,996 is hereby incorporated herein by reference.

U.S. Pat. No. 8,172,752 issued to Russ on 8 May 2012, entitled, “Automatic Wireless PAN/LAN Switching,” discloses a communication system used by a portable patient monitoring device for connecting to other devices, including a docking station, via one or more networks. The communications system includes an adaptive communication interface which, when the patient monitoring device is docked, establishes communication with a first network, and receives from a first docking station a first unique identifier that identifies the first docking station, allowing the patient monitoring device to communicate patient parameters to the first destination. While the portable patient monitoring device is undocked, the adaptive communication interface is inhibited from establishing communication with a network providing an identifier to the portable patient monitor that does not match the first unique identifier. Upon docking to a second docking station, the patient monitoring device establishes communication with a second network using a new unique identifier received from the second docking station via a second exclusive communication link. The entirety of the U.S. Pat. No. 8,172,752 is hereby incorporated herein by reference.

U.S. Patent Application Pub. No. US 2005/0192845 (Ser. No. 10/908,372) filed by Brinsfield et al. on 9 May 2005 (published 1 Sep. 2005), entitled “Mobile Clinical Information System,” discloses a wireless bi-directional portable patient monitor incorporated into a mobile clinical information management system. The portable patient monitor includes a communications interface to receive patient data from a wireless local area network (WLAN) within a medical care facility and transmit care parameters as needed to the wireless network (WLAN) in response. The portable patient monitor includes a processor connected to the communications interface to process the patient data and the care parameters. A display is connected to the processor to display the processed patient data to the health care provider. The monitor includes an input device connected to the processor to allow a change in the care parameters by the health care provider. The portable patient monitor is also configured to allow wireless transport on the health care provider for extended periods. The mobile clinical information management system includes a number of bedside patient monitors to connect to the patients and transmit the patient data. The system also includes the wireless network coupled to the bedside patient monitors and the portable patient monitors to improve efficiencies in the delivery of health care in the medical care facility. The entirety of U.S. Patent Application Pub. No. US 2005/0192845 (Ser. No. 10/908,372) is hereby incorporated herein by reference.

U.S. Patent Application Pub. No. US 2006/0064020 (Ser. No. 10/944,982) filed by Burnes et al. on 20 Sep. 2004 (published 23 Mar. 2006), entitled “Clinical Dashboard Monitor,” discloses a remote patient monitoring system including a graphical user interface (GUI) that displays a summary table of categorized parameter values for multiple patients simultaneously. The remote patient monitoring system further includes a central database for receiving data from remote medical devices via a communications network and a processor for parameterizing and categorizing summary data to be displayed by the GUI. The displayed summary parameter values are formatted according to category to allow a parameter value category to be visually recognized. In one embodiment, parameter values are categorized according to a need for clinical attention such that a clinician may view a summary table of categorized parameter values and recognize which patients may require clinical attention as indicated by the formatted parameter values. The entirety of U.S. Patent Application Pub. No. US 2006/0064020 (Ser. No. 10/944,982) is hereby incorporated herein by reference.

Notably, none of the foregoing prior art contains a method, device, or system by which the neurobehavioral status of a patient being monitored may be measured. There is therefore a long-felt need for being able to measure the neurobehavioral status of a patient being monitored by a distributed patient monitoring system. Among its several aims and objectives, the presently disclosed invention seeks to fill this need.

Background to Neurobehavioral Performance

Aspects of the presently disclosed invention relate to various features and details of neurobehavioral performance. Broadly defined, “neurobehavioral performance” refers to an individual's ability to perform a specific task that requires one or more cognitive functions that rely on alertness level and/or fatigue state. (The terms “neurobehavioral performance” and “neurobehavioral status” shall be used synonymously and interchangeably herein.) Such cognitive functions include (without limitation) concentration, short-term or long-term memory, visual or other sensory acuity, alertness, gross motor dexterity, fine motor skill, and/or the like. As used herein, the terms (used interchangeably) “neurobehavioral performance prediction(s),” “predicted neurobehavioral performance,” and “predicted neurobehavioral performance level(s)” refer to the output of a biomathematical model capable of modeling and/or predicting neurobehavioral performance states when given appropriate inputs. Non-limiting factors that may impact a subject's neurobehavioral performance include: sleep disruption, sleep restriction, circadian misalignment, sleep inertia, extended task performance, extended work/duty hours, multitasking, (extended) physical exertion, psychological stresses (e.g., time pressure; family, financial, or legal issues etc.), environmental stressors (e.g., extreme temperature or humidity conditions, ambient noise, ambient vibration, ambient light conditions, altitude “hypoxia” etc.), certain medical conditions or behavioral disorders (e.g., Parkinson's, Alzheimer's, dementia, or any age-related brain dysfunction or mild cognitive impairment, brain injuries, mood disorders, and certain psychoses, etc.).

Methods to Test Neurobehavioral Performance Generally

The presently disclosed invention may make use of any methods or techniques used to measure neurobehavioral performance. Such methods and techniques may include context-relative performance tasks, such as a workplace-specific task (e.g., assembling X number of specific product units in a particular factory in time T and/or the like), standardized line-of-work specific tasks (e.g., typing a standard document within an acceptable accuracy threshold on standard equipment, and/or the like), and so-called “special tasks” that highlight particular neurobehavioral performance characteristics (e.g., executing a specific complex driving, flying, or navigation maneuver within an acceptable threshold, navigating a standardized obstacle course on foot, assembling a particular standardized complex manufactured object, and/or the like). Performance measures for such neurobehavioral tasks may come from direct human observation, measurement instruments, or from embedded systems. Furthermore, performance assessment on one or more neurobehavioral tasks may be measured by one or more standard tests including but not limited to: the Psychomotor Vigilance Test (PVT), the Motor Praxis Test (MPraxis), the Visual Object Learning Test (VOLT), the Fractal-2-Back Test (F2B), the Conditional Exclusion Task (CET), the Matrix Reasoning Task (MRsT), the Line Orientation Test (LOT), the Emotion Recognition Task (ER), the Balloon Analog Risk Task (BART), the Digit Symbol Substitution Test (DSST), the Forward Digit Span (FDS), the Reverse Digit Span (BDS), the Serial Addition and Subtraction Task (SAST), the Go/NoGo Task, the Word-Pair Memory Task, the Word Recall Test (Learning,Recall), the Motor Skill Learning Task, the Threat Detect Task, the Descending Subtraction Task (DST), the Positive Affect Negative Affect Scales-Extended version (PANAS-X) Questionnaire, the Pre-Sleep/Post-Sleep Questionnaires for astronauts, the Beck Depression Inventory (BDI), the Conflict Questionnaire, Karolinska Drowsiness Test (KDT), the Visual Analog Scales (VAS), the Karolinska Sleepiness Scale (KSS), the Profile of Mood States Long/Short Form Questionnaire (POMS/POMS SF), the Stroop Test, and/or the like.

Methods to Test Fatigue Specifically

Although the presently disclosed invention may be used to monitor a subject's neurobehavioral performance status generally, particular embodiments are specifically directed to the monitoring of a subject's fatigue or alertness state. (As used herein the terms “alertness” and “fatigue” shall refer to the same neurobehavioral characteristic, but from “opposite” perspectives—i.e., alertness a is inversely correlated to f.) Embodiments of the presently disclosed invention may make use of one or more techniques for measuring or testing an individual's alertness or fatigue levels (referred to hereinafter as “fatigue-measurement techniques”). Particular embodiments of the invention are sufficiently adaptable to utilize many (if not all) of these known fatigue-measurement techniques. Non-limiting and non-mutually exclusive examples of suitable fatigue-measurement techniques which may be used in various embodiments of the invention include testing techniques which use: (i) objective reaction-time tasks, stimulus-response tests, and cognitive tasks such as the Psychomotor Vigilance Task (PVT) or variations thereof (Dinges, D. F. and Powell, J. W. “Microcomputer analyses of performance on a portable, simple visual RT task during sustained operations” Behavior Research Methods, Instruments, & Computers 17(6): 652-655, 1985) and/or a so-called digit symbol substitution test; (ii) subjective alertness, sleepiness, or fatigue measures based on questionnaires or scales such as (without limitation) the Stanford Sleepiness Scale, the Epworth Sleepiness Scale (Jons, M. W., “A new method for measuring daytime sleepiness—the Epworth sleepiness scale” Sleep 14 (6): 54-545, 1991), and the Karolinska Sleepiness Scale (Åkerstedt, T. and Gillberg, M. “Subjective and objective sleepiness in the active individual” International Journal of Neuroscience 52: 29-37, 1990); (iii) EEG measures and sleep-onset-tests including (without limitation) the Karolinska drowsiness test (Åkerstedt, T. and Gillberg, M. “Subjective and objective sleepiness in the active individual” International Journal of Neuroscience 52: 29-37, 1990), Multiple Sleep Latency Test (MSLT) (Carskadon, M. W. et al., “Guidelines for the multiple sleep latency test—A standard measure of sleepiness” Sleep 9 (4): 519-524, 1986) and the Maintenance of Wakefulness Test (MWT) (Mitler, M. M., Gujavarty, K. S. and Browman, C. P., “Maintenance of Wakefulness Test: A polysomnographic technique for evaluating treatment efficacy in patients with excessive somnolence” Electroencephalography and Clinical Neurophysiology 53:658-661, 1982); (iv) physiological measures such as (without limitation) tests based on blood pressure and heart rate changes, and tests relying on pupillography and/or electrodermal activity (Canisius, S, and Penzel, T., “Vigilance monitoring—review and practical aspects” Biomedizinische Technik 52(1): 77-82., 2007); (v) embedded performance measurement systems, devices, and processes such as (without limitation) devices that are used to measure a driver's performance in tracking the lane marker on the road (see, e.g., U.S. Pat. No. 6,894,606); and (vi) simulators that provide a virtual environment to measure specific task proficiency such as commercial airline flight simulators (Neri, D. F., Oyung, R. L., et al., “Controlled breaks as a fatigue countermeasure on the flight deck” Aviation Space and Environmental Medicine 73(7): 654-664, 2002); and/or (vii) the like. Particular embodiments of the invention may make use of any one or more of the fatigue-measurement techniques described in the aforementioned references or various combinations and/or equivalents thereof. All of the publications referred to in this paragraph are hereby incorporated by reference herein.

Models for Predicting Neurobehavioral Performance

The presently disclosed invention is designed to utilize any biomathematical model designed generally to model any one or more of human subject's neurobehavioral performance characteristics. Such biomathematical models are referred to herein as “neurobehavioral performance models.” Particular embodiments are specifically designed to utilize biomathematical models that model a human subject's alertness and/or fatigue state. Such biomathematical models are referred to herein as “fatigue models.” As used herein, the terms “biomathematical model(s),” “neurobehavioral performance model(s),” and “fatigue model(s)” shall have the following relationship: fatigue models are a subset of neurobehavioral performance models (fatigue and/or alertness being a type of neurobehavioral performance), and neurobehavioral performance models are, in turn, a subset of biomathematical models.

Among the neurobehavioral performance models utilized by the presently disclosed invention, particular embodiments may utilize the so-called “two-process model” of sleep regulation developed by Borbèly et al. in 1999. The Borbèly two-process model posits the existence of two primary regulatory mechanisms: (i) a sleep/wake-related mechanism that builds up exponentially during the time that the subject is awake and declines exponentially during the time that the subject is asleep, and is called the “homeostatic process” or “process S;” and (ii) an oscillatory mechanism with a period of (nearly) 24 hours, called the “circadian process” or “process C.” Without wishing to be bound by theory, the circadian process has been demonstrated to be orchestrated by the suprachiasmatic nuclei of the hypothalamus. The neurobiology of the homeostatic process is only partially known and may involve multiple neuroanatomical structures. Total alertness at a given time y(t), which is one non-limiting example of neurobehavioral performance, may then be represented as a sum of the C and S processes (see Equation 3, below).

Further details of the Borbèly two-process fatigue model are contained in PCT published patent application Systems and Methods for Individualized Alertness Predictions, inventors Mott C. G., Mollicone, D. J., et al., WIPO publication No. WO 2009/052633, the entirety of which is incorporated herein by reference and from which portions of the following discussion are excerpted for convenience and clarity.

Specifically, in accordance with the two-process model, the circadian process C may be represented by:

$$C ( t ) = γ {\sum }_{l = 1}^{5} {a}_{l} sin ( 2 l π ( t - ϕ ) / τ ) \qquad ( 1 )$$

where t denotes clock time (in hours, e.g. relative to midnight), φ represents the circadian phase offset (i.e. the timing of the circadian process C relative to clock time), γ represents the circadian amplitude, and τ represents the circadian period which may be fixed at a value of approximately or exactly 24 hours. The summation over the index/serves to allow for harmonics in the sinusoidal shape of the circadian process. For one particular application of the two-process model for alertness prediction, l has been taken to vary from 1 to 5, with constants a₁ being fixed at a₁=0.97, a₂=0.22, a₃=0.07, a₄=0.03, and a₅=0.001.

The homeostatic process S may be represented by:

$$S ( t ) = \{ \begin{matrix}{e}^{- {ρ}_{w} Δ t} {S}_{t - Δ t} + ( 1 - {e}^{- {ρ}_{w} Δ t} ) & if during wakefulness \\ {e}^{- {ρ}_{w} Δ t} {S}_{t - Δ t} & if during sleep\end{matrix} \qquad \begin{matrix}( 2 a ) \\ ( 2 b )\end{matrix}$$

(S>0), where t denotes (cumulative) clock time, Δt represents the duration of time step from a previously calculated value of S, ρ_{W} represents the time constant for the build-up of the homeostatic process during wakefulness, and ρₛ represents the time constant for the recovery of the homeostatic process during sleep.

Given equations (1), (2a) and (2b), the total alertness according to the two-process model may be expressed as a sum of: the circadian process C, the homeostatic process S multiplied by a scaling factor κ, and an added noise component ε(t):

y(t)=κS(t)+C(t)+ε(t) (3)

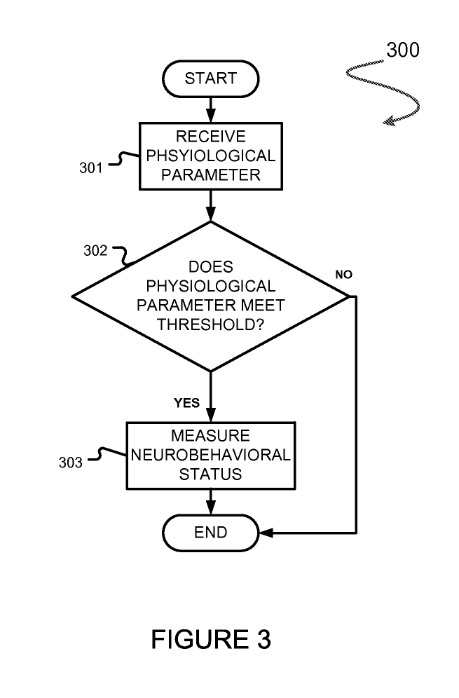

Furthermore, it is useful to be able to describe the homeostatic process S for test subject after one or more transitions between being asleep and being awake. The sleep-wake transitions are commonly (but without limitation) represented as square wave signals oscillating between the binary states of being asleep (value=1 herein, without limitation) and being awake (value=0 herein, without limitation), referred to as sleep functions. As discussed in connection with FIG. 3B, other mathematical representations of sleep status and effectiveness can be utilized by the presently disclosed invention.

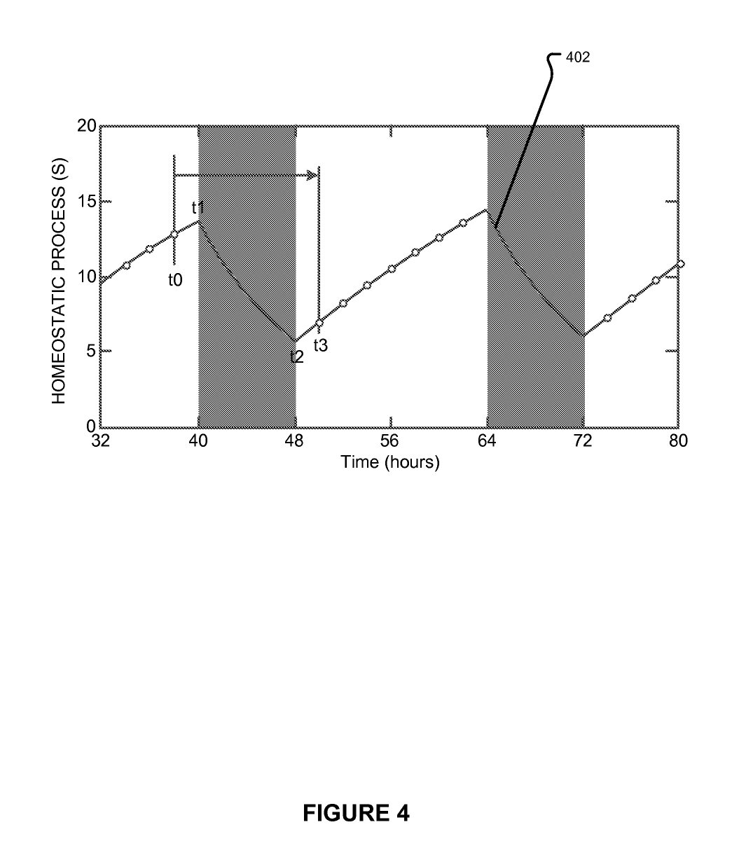

As described in more particular detail below, the systems and methods of the invention may make use of measured neurobehavioral performance levels which is typically only available when the subject is awake. Consequently, it may be desirable to describe the homeostatic process between successive periods that the test subject is awake. As the circadian process C is independent from the homeostatic process S, we may consider as an illustrative case of neurobehavioral performance using only the homeostatic process S of equations (2a), (2b). Consider the period between t₀ and t₃ shown in FIG. 4. During this period, the subject undergoes a transition from awake to asleep at time t₁ and a transition from asleep to awake at time t₂. Applying the homeostatic equations (2a), (2b) to the individual segments of the period between t₀ and t₃ yields:

S(t₁)=S(t₀)e^{-ρ}^{_{w}}^{T}^{₁}+(1−e^{-ρ}^{_{w}}^{T}^{₁}) (4a)

S(t₂)=S(t₁)e^{-ρ}^{ₛ}^{T}^{₂} (4b)

S(t₃)=S(t₂)e^{-ρ}^{_{w}}^{T}^{₃}(1−e^{-ρ}^{_{w}}^{T}^{₃}) (4c)

Where

T₁=t₁−t₀ (5a)

T₂=t₂−t₁ (5b)

T₃=t₃−t₂ (5c)

Substituting equation (5a) into (5b) and then (5b) into (5c) yields an equation for the homeostat at a time t₃ as a function of an initial known homeostat condition S(t₀), the time constants of the homeostatic equations (ρ_{w}, ρₛ) and the transition durations (T₁, T₂, T₃):

$$\begin{matrix}S ( {t}_{3} ) = {f}_{s} ( S ( {t}_{0} ) , {ρ}_{w} , {ρ}_{s} , {T}_{1} , {T}_{2} , {T}_{3} ) \\ = [ S ( {t}_{0} ) {e}^{- {ρ}_{w} {T}_{1}} + ( 1 - {e}^{- {ρ}_{w} {T}_{1}} ) ] {e}^{- {ρ}_{s} {T}_{2} - {ρ}_{w} {T}_{3}} + ( 1 - {e}^{- {ρ}_{w} {T}_{3}} )\end{matrix} \qquad ( 6 )$$

Equation (6) applies to the circumstance where t₀ occurs during a period when the test subject is awake, there is a single transition between awake and asleep at t₁ (where t₀<t₁<t₃), there is a single transition between asleep and awake at t₂ (where t₁<t₂<t₃), and then t₃ occurs after the test subject is awake again.

Additional fatigue models may be utilized by particular embodiments. Other non-limiting examples of fatigue models include Akerstedt's “three-process model of alertness” (see, e.g., Akerstadt, T., et al. “Predictions from the Three-Process Model of Alertness,” Aviation, Space, and Environmental Medicine, 75:No. 3, §II (March 2004); see also Akerstedt, T. et al. “A Model of Human Sleepiness,” excerpted from Sleep '90 J. Horne, Ed. (Pontenagel Press 1990)); Achermann's “two-process model revisited” (see e.g., Achermann, P., “The Two-Process Model of Sleep Regulation Revisited,” Aviation, Space, and Environmental Medicine, 75:No. 3, §II (March 2004)); Avinash's “process-U model” (see Avinash, D., “Parameter Estimation for a Biomathematical Model of Psychomotor Vigilance Performance under Laboratory Conditions of Chronic Sleep,” Sleep-Wake Research in the Netherlands 16:39-42 (Dutch Society for Sleep-Wake Research 2005); Beersma's “modified two-process model” (see, e.g., Beersma, D. G. M., “Models of Human Sleep Regulation,” Sleep Medicine Reviews 2:No. 1, pp. 31-43 (W.B. Saunders Co. Ltd. 1998)); Belyavin and Spencer's “QinetiQ Approach” (see, e.g., Belyavin, A. J. and Spencer, M. B., “Modeling Performance and Alertness: the QinetiQ Approach,” Aviation, Space, and Environmental Medicine, 75:No. 3, §II (March 2004)); the “circadian alertness simulator” (see, e.g., Dijk, D. J., et al. “Fatigue and Performance Models: General Background and Commentary on the Circadian Alertness Simulator for Fatigue Risk Assessment in Transportation,” Aviation, Space, and Environmental Medicine, 75:No. 3, §II (March 2004)); the so-called “new model class” (see, e.g., McCauley, P., et al, “A new mathematical model for the homeostatic effects of sleep loss on neurobehavioral performance,” Journal of Theoretical Biology, 256:227-239 (Reed-Elsevier 2009)); alternative models such as nonparametric approaches and neural networks (see, e.g., Reifman, J., “Alternative Methods for Modeling Fatigue and Performance,” Aviation, Space, and Environmental Medicine, 75:No. 3, §II (March 2004)); and/or the like. Particular embodiments of the presently disclosed invention may make use of any one or more of the biomathematical models described in the aforementioned references or various combinations and/or equivalents thereof. All of the publications referred to in this paragraph are hereby incorporated by reference herein.

The presently disclosed invention may utilize one or more of the foregoing biomathematical models to predict neurobehavioral performance levels when certain inputs are provided. Particular embodiments may focus on fatigue and/or alertness as the specific neurobehavioral characteristic being measured and/or assessed.

### THE FIGURES

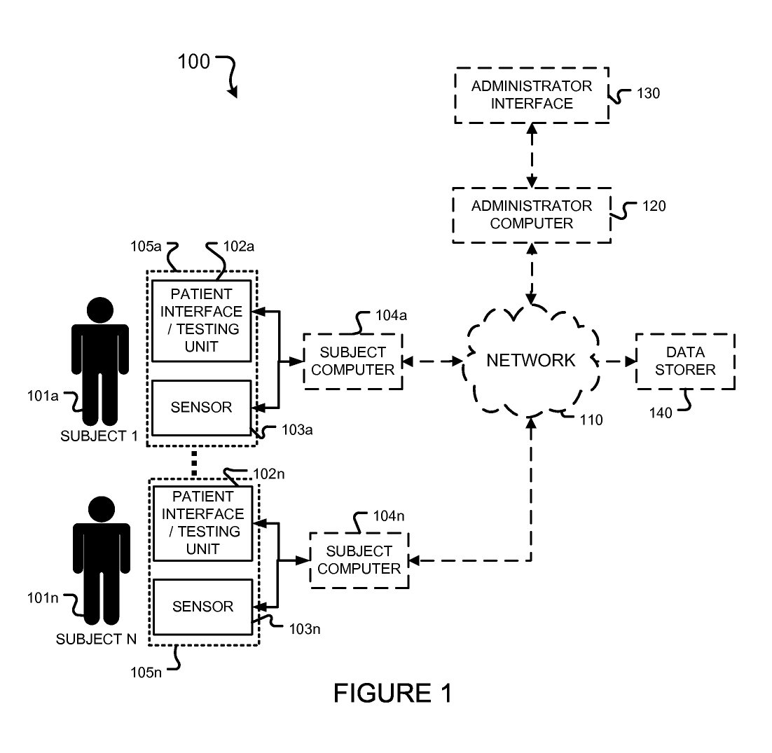

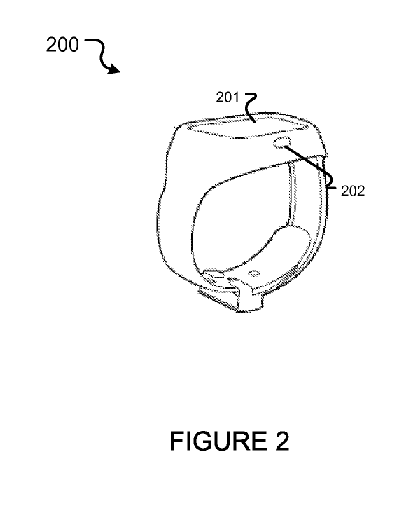

FIG. 1 provides a component-level diagram of a system 100 for monitoring the physiological and neurobehavioral status of one or more individuals (including, without limitation, medical patients), in accordance with particular embodiments. System 100 comprises a patient interface 102a, 102n for each patient 101a, 101n being monitored. Patient interface 102a, 102n may comprise any device capable of applying a neurobehavioral test to patient 101a, 101n, such as a portable computing device, task-specific dedicated device a mobile monitoring device, a mobile phone, a mobile personal data/digital assistant (PDA), and/or the like. FIG. 2 provides a non-limiting example of a task-specific dedicated device that is capable of functioning as testing unit 102a, 102n.

System 100 also comprises at least one physiological sensor 103a, 103n associated with each patient 101a, 101n being monitored. Physiological sensor 103a, 103n is any device capable of reading a physiological parameter from a human subject including subject 101a, 101n, and may comprise (without limitation) of a blood pressure gauge; a hear rate monitor; a heart rate monitor with heart-rate variability measurement capability; EEG/EKG machine, system or device; biorhythm measurement devices, heat balance detectors, perspiration monitors, and/or the like. In particular embodiments physiological sensors 103a, 103n are separate components from testing units 102a, 102n. In other embodiments sensors 103a, 103n and units 102a, 102n are integrally connected and form a single unit 105a, 105n.

Particular embodiments of system 100 take advantage of networked computing operability. As such, one or more of the following optional components may be present: subject computer 104a, 104n, communications network 110, administrator computer 120, administrator interface 130, and data storage unit 140. Subject computer 104a, 104n serves to provide client-side functionality, such as displaying output from physiological sensors 103a, 103n, providing an additional interface for neurobehavioral status testing along with testing unit 102a, 102, storing data and/or results associated with each of these devices 102a, 102n, 103a, 103n, and/or the like. Subject computer 104a, 104n may be any suitable computer including (without limitation) a personal computer, a laptop computer, a mobile computer, a dedicated computing device, an embedded system, a personal data assistant, a mobile, phone, and/or the like. In particular embodiments, subject computer 104a, 104n may also be connected to a communications network 110, which may comprise a WAN, LAN, intranet, or internet (including the Internet), and/or the like. Connection between subject computer 104a, 104n and communications network 110 may be through any suitable technology known in the art including a wireless connection and/or the like. Additionally, in yet other embodiments, an administrative computer 120 may be connected to communications network 110. Administrative computer 120 may provide administrative features such as system control functionality, neurobehavioral test and/or physiological parameter analysis, and/or the like. Administrative computer may comprise any suitable computing device including (without limitation) a server, a mainframe computer, a personal computer, a laptop computer, a mobile computer, a dedicated computing device, an embedded system, a personal data assistant, a mobile, phone, and/or the like. Administrator computer 120 may also be provisioned with an administrator interface 130 for providing output to and receiving input from an administrative user (not shown). Administrative interface may be any suitable interface for such purposes, including (without limitation) a graphical user interface, a text interface, a pointer-device enabled interface, a touchscreen interface, an LED display interface, and/or the like. Particular embodiments may also be equipped with a data storage unit 140, connected to the communications network 110. Data storage unit 140 may be any device or component used to store data as is known in the art. The aforementioned networked embodiments of system 100 may be based upon the network architecture, components, systems, and/or other teachings found in one or more of aforementioned U.S. Pat. Nos. 5,579,775, 5,752,917, 6,553,262, 6,659,947, 6,749,566, 7,256,708, 7448,996, and 8,172,752 and U.S. Patent Application Pub. Nos. 2005/0192845 and 2006/0064020, or their equivalents or variations thereof as are known in the art, in accordance with particular embodiments.

FIG. 2 illustrates a non-limiting example of device 200, which integrally combines a testing unit with a physiological sensor, in accordance with particular embodiments. One non-limiting embodiment of a device 200 is a wrist-worn device that monitors physical motion, such as an actigraph. In addition, device 200 may record light levels, on-wrist/off-wrist state, heart rate, or other physiological variables. Device 200 may process measured signals to generate interpreted values such as motion sensor zero-crossing metrics, and activity/sleep state.

In particular embodiments, device 200 may be used for single unit 105a, 105n, which provides functionality for both neurobehavioral status assessment and physiological sensing. (In other embodiments, it may be used strictly for testing unit 102a, 102n, without providing any physiological sensing capabilities.) Device 200 may optionally contain memory that stores recently collected data and a communicator that transfers data to a computer. The communicator may be a wireless transceiver (e.g. Bluetooth, Zigby, wireless USB), or a wired transceiver (e.g. USB, CAN or other electrical signal protocol). As non-limiting examples the computer may comprise a personal computer located in near proximity to a location that the individual may be present at one or more times (e.g. in the individual's home), a mobile phone, a personal digital assistant, a special-purpose data transfer device, or a computer embedded inside the sensor housing.

To administer a neurobehavioral test, device 200 has a display screen 201 and a user-input button 202. Display screen 201 is capable of providing a stimulus to subject 101a, 101n, such as a visual stimulus, and button 202 is capable of receiving a response input from subject 101a, 101n. In particular embodiments, device 200 is also capable of receiving one or more physiological parameters from subject 101a, 101n, such as heart rate, temperature, and/or the like.

Systems for transferring data from the sensor to the computer may include a wireless data transmitter with that can be configured in a low power operation mode, a searching for a connection to a wireless data receiver mode, and a transmitting data with a wireless data receiver mode. Transition to the searching for a connection mode may occur at connection polling interval. The connection polling interval may be automatically triggered at a fixed duration, initiated by the sensor or a by a controller. In one embodiment with a bluetooth communicator in the sensor, the communicator may be enabled at fixed intervals (e.g. every 30 seconds) and attempt to establish a connection to a computer. If a connection while the wireless transmitter is in searching for a connection mode, then the sensor may transmit data that has not yet been confirmed uploaded to the computer. Following successful data transmission, including confirmation handshaking with the computer the sensor may mark the transmitted data as successfully uploaded or may delete the data from its local memory. In between communication-establishing polling attempts, the wireless transmitter may advantageously enter a low-power mode to reduce energy consumption. The communication-establishing polling interval may be fixed at a rate determined by a configuration setting. In some embodiments the communication-establishing polling interval may selected based on the output of an interval objective function. The interval objective function may be based in part on battery size, remaining energy in a battery, projected power consumption rate, size of memory store, rate of memory usage, desired operational run-time, a frequency weighting parameter for prioritizing frequency of data transmission, a run-time weighting parameter for prioritizing achieving the desired run-time, energy consumption rate for communication-establishing polling, energy consumption rate for data transfer, energy consumption rate for data recording, a preferred polling interval, time-of-day weighting parameter, a maximum polling interval, and a minimum polling interval. In some embodiments the objective function may be recalculated by a controller at one or more times, and the next polling interval may be updated. In some embodiments the communication-establishing polling interval may be determined based on one or more the following non-limiting factors: default to a polling interval configuration setting, decrease the interval if projected energy reserves will exceed the desired operational lifetime, decrease the interval if the memory utilized is approaching the maximum available, increase the rate if project energy reserve will not exceed the desired operational lifetime, increase the interval if the memory utilized is not approaching the maximum available. The polling interval may have a mode in which it is triggered manually. One embodiment of a system to activate a manual communication establishment polling is shown in FIG. 2, in which a button 202 is included as part of a wrist-worn sensor (in the form of device 200). If a user presses the button 202 then the device 200 may activate polling. The manual trigger polling mode may also be combined with other polling modes in an additive manner. The polling interval may have a mode in which polling is initiated by the patient interface. The patient interface polling mode may be combined with other polling modes in an additive manner.

An interface 201 on the device 200 and/or subject computer 104a, 104n may provide indications of the communication establishment polling mode, the communication transmission state (e.g. inactive, polling for communication connection, transmitting data), transitions between communication transmission states (e.g. transition from transmitting data to inactive as a data transmission is complete), memory fullness state, and energy reserve state. Indicators may include graphical (e.g. LCD display), auditory (e.g. microphone), or tactile systems (such as haptic feedback).

The subject computer 104a, 104n may have a communication connection to a network 100 using systems such as a Wi-Fi 802.11 transceiver, an ethernet transceiver or other local area, or wide area networking devices. The subject computer 104a, 104n may transfer data that is received from the device 200 over a network (e.g. using internet transmission protocol) to a data storer 140. Such a transfer may be performed immediately upon receipt of data from a sensor device (such as device 200), or at some other interval determined by availability of a network connection or configured upload frequencies. The data transferred to the data storer 140 may include information about the patient and patient equipment configuration. The data may be encrypted.

The patient interface (not shown) may comprise a graphical interface (such as display 201) and human input device (such as button 202) connected to the patient computer (e.g. an LCD display and keyboard) or may be integrated into a single device with the computer (e.g. laptop, mobile phone). The patient interface may present individuals with computerized cognitive tests. Tests may comprise, but are non-limited to objective reaction-time tasks and cognitive tasks such as the Psychomotor Vigilance Task (PVT) or variations thereof (Dinges, D. F. and Powell, J. W. “Microcomputer analyses of performance on a portable, simple visual RT task during sustained operations.” Behavior Research Methods, Instruments, & Computers 17(6): 652-655, 1985) or the Digit Symbol Substitution Task. The patient interface may provide means for the individual to review data from cognitive tests and/or sensors. Data may be reviewed that is present in the memory of the patient computer, or may be retrieved through the computer from the data storer. Examples of such data review include comparing current cognitive test performance against historical results, or comparing recent sleep time data from an actigraph to historical sleep time data. The patient interface may provide predictions of future performance to the individual based on predictions calculated by the patient computer or stored in the data storer 140. The patient interface may provide means for the individual to enter information such as demographic or behavioral questions (e.g. recent sleep history). The patient interface may promp the individual to initiate a data transfer polling when the device 200 is configured with a manually initiated polling mode. In such cases, the patient interface may require the individual to successfully complete a data transfer prior to continuing with another function, and could verify this by communication with the computer. In other cases the patient interface may request the individual to initiate a data transfer polling but not require successful completion before continuing with other tasks.

The data storer 140 may record data from multiple patients 101a, 101n, allowing secure uploading from multiple distributed locations over a network 110. Permissioned access to the data store 140 may allow patient computers 104a, 104n to upload data and/or retrieve data for the specific patient 101a, 101n, and may allow administrator computers 120 to access data from multiple patients 101a, 101n. The data store 140 may also store configuration settings that may be retrieved by patient computers 104a, 104n. When patient computers 104a, 104n connect to the data storer 140, they may download new configuration settings that are present in the data storer 140, and the configuration settings may then be passed to the sensor device 200 and/or patient interface (not shown).

The administrator interface 130 may provide a means for individuals to monitor the status of one or more patients 101a, 101n through access to the data store 140. In the embodiment shown in FIG. 1 the administrator interface 130 accesses data from the data storer 140 over a central network 100 (e.g. the Internet, and/or the like). In other embodiment the administrator interface 130 could connect to the data storer 140 on a separate network or through direct communication (not shown). The administrator interface 130 may provide a graphical display that shows data upload history of one or more patients 101a, 101n, errors that have been logged by patient computers 104a, 104n, data recorded from sensors 103a, 103n and patient interfaces (not shown), and configuration settings of the patient computers 104a, 104n, sensors 103a, 103n and patient interfaces (not shown). The adminstrator interface 130 may provide a means to upload new configuration settings for one or more patient computers to the data storer 140.

In some embodiments the administrator interface 130 and patient interface (not shown) may be provided as web-based interfaces server over the network by a server. The server may be the data storer 140, an administrator computer 120 or a patient computer 104a, 104n.

In some embodiments the data storer 140, administrator computer 120, or patient computer 104a, 104n may calculate personalized data interpretations based on historical sensor data and patient interface data. Such interpretations may include calculations of individual cognitive states (e.g. sleep debt), individual cognitive traits (e.g. susceptibility to sleep loss), or normative cognitive performance values (e.g. percentage below or above population norms on cognitive performance tests). Calculated values may be stored in the data storer and may available to through the administrative interfaces and/or patient interfaces. Other interpretations may include predictions of individual future cognitive states based in part on future behavior of individuals (e.g. future sleep schedules input by individuals through the patient interface or input from a work roster) or in part on past cognitive performance and physiological state assessed from the patient interface data on the sensor data.

FIG. 3 provides a flowchart of a method 300 for using device 200 (or an equivalent arrangement of suitable components) as part of system 100 for monitoring the physiological and neurobehavioral status of subject 101a, 101n, in accordance with particular embodiments. Method 300 commences in step 301 in which at least one physiological parameter is received from subject 101a, 101n. Physiological parameter may be any measured physiological quantity known in the art. Method 300 proceeds to step 302, in which the step-301 received physiological parameter is compared to a threshold. If the step-301 received physiological parameter meets the threshold condition, process control proceeds to step 303, wherein a measurement is taken of then neurobehavioral status of the subject 101a, 101n, and the process then ends (or repeats). Step-303 measured neurobehavioral status may be any neurobehavioral status known in the art, and the techniques, devices, methods, or systems used to measure a neurobehavioral status of subject 101a, 101n in step 303 may also be of any type known in the art. If the threshold condition is not met is step, 302, the process ends (or repeats) without executing step 303.

Certain implementations of the invention comprise computers and/or computer processors which execute software instructions which cause the processors to perform a method of the invention. For example, one or more processors in a system may implement data processing blocks in the methods described herein by executing software instructions retrieved from a program memory accessible to the processors. The invention may also be provided in the form of a program product. The program product may comprise any non-transitory medium which carries a set of computer-readable instructions that, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs and DVDs, electronic data storage media including ROMs, flash RAM, or the like. The instructions may be present on the program product in encrypted and/or compressed formats.

Certain implementations of the invention may comprise transmission of information across networks, and distributed computational elements which perform one or more methods of the inventions. Such a system may enable a distributed team of operational planners and monitored individuals to utilize the information provided by the invention. A networked system may also allow individuals to utilize a graphical interface, printer, or other display device to receive personal alertness predictions and/or recommended future inputs through a remote computational device. Such a system would advantageously minimize the need for local computational devices.

Certain implementations of the invention may comprise exclusive access to the information by the individual subjects. Other implementations may comprise shared information between the subject's employer, commander, flight surgeon, scheduler, or other supervisor or associate, by government, industry, private organization, and/or the like, or by any other individual given permitted access.

Certain implementations of the invention may comprise the disclosed systems and methods incorporated as part of a larger system to support rostering, monitoring, selecting or otherwise influencing individuals and/or their environments. Information may be transmitted to human users or to other computerized systems.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e. that is functionally equivalent), including components that are not structurally equivalent to the disclosed structure which performs the function in the illustrated exemplary embodiments of the invention.

As will be apparent to those skilled in the art in the light of the foregoing disclosure, many alterations and modifications are possible in the practice of this invention without departing from the spirit or scope thereof. For example:
The systems and methods of various embodiments may be extended to include other measures of human performance such as gross-motor strength, dexterity, endurance, or other physical measures. For example, fatigue may be replaced by one or more other types of neurobehavioral performance such as “sleepiness”, “alertness”, “tiredness”, “cognitive performance”, “cognitive throughput”, and/or the like.
Other models or estimation procedures may be included to deal with biologically active agents, external factors, or other identified or as yet unknown factors affecting alertness/fatigue.

What is claimed is:

1. A system for monitoring a physiological status of an individual, the system comprising:
a physiological sensor that outputs one or more physiological measurements indicative of a subject's physical motion when worn by the subject;
a wireless data transmitter that can be configured in a low power operation mode, a searching for a connection to a wireless data receiver mode, and a transmitting data with a wireless data receiver mode;
a memory store that saves one or more physiological measurements;
a controller connected to the physiological sensor, the memory store, and the wireless data transmitter, and configured to:
transition the wireless data transmitter from the low power operation mode to the searching for a connection mode at a connection polling interval;
transition the wireless data transmitter from the searching for a connection mode to the transmitting data mode if a connection is made or transition to the low power mode if a connection is not made;
save physiological measurements to the memory store when the wireless data transmitter is not in the transmitting data mode;
determine a connection polling interval based on an interval objective function.

2. A system according to claim 1 wherein the interval objective function is based at least in part on an operational performance parameter, and a device status parameter

3. A system according to claim 2 wherein the operational performance parameter is one or more of: desired operational run-time, a frequency weighting parameter for prioritizing frequency of data transmission, a run-time weighting parameter for prioritizing achieving the desired run-time, a preferred polling interval, time-of-day weighting parameter, a maximum polling interval, and a minimum polling interval.

4. A system according to claim 2 further comprising a battery that provides power to the system components, and wherein the device status parameter is one of: battery capacity, remaining energy in a battery, projected power consumption rate, energy consumption rate for communication-establishing polling, energy consumption rate for data transfer, energy consumption rate for data recording.

5. A system according to claim 2 wherein the device status parameter is one of: capacity of memory store, and rate of memory usage.

6. A system according to claim 2 wherein the interval objective function is based at least in part on more than one operational performance parameters.

7. A system according to claim 2 wherein the interval objective function is based at least in part on more than one device status parameters.

8. A system according to claim 1, wherein the controller determining the connection polling interval further comprises a microprocessor.

9. A system according to claim 1 wherein the physiological sensor comprises an acceleration sensor.

10. A system according to claim 9 wherein the physiological measurement is an actigraphy activity count determined based on data from the physiological sensor output.

11. A system according to claim 1 further comprises one or more additional physiological sensors.

12. A system according to claim wherein the one or more additional physiological sensors comprise one or more of: a blood pressure gauge, a hear rate monitor, a heart rate monitor with heart-rate variability measurement capability, EEG/EKG system, biorhythm measurement devices, heat balance detectors, and a perspiration monitor.

13. A system according to claim 1 wherein the controller is further configured to:
sample the physiological sensor at a fixed sampling rate,
receive one or more physiological sensor measurements from the sampled physiological sensors, and
store the one or more received physiological sensor measurements in the memory store.

14. A system according to claim 1 wherein the wireless data transmitter uses a Bluetooth compatible protocol.

15. A system according to claim 1 wherein the wireless data transmitter can transmit and received data.

16. A system according to claim 1 wherein the controller is further configured to read data from the memory store and transmit data over the wireless data transmitter in a last-in first-out sequence.

17. A system according to claim 1 wherein the wireless data transmitter can transmit and received data.

18. A system according to claim 1 further comprising an administrative user interface.

19. A system according to claim 19, wherein the wireless data transmitter can receive an update from the administrative user interface.

20. A system according to claim 19, wherein the update received from the administrative user interface may comprise one or more of:
an instruction to transition the wireless data transmitter from a low power operation mode to a searching for connection mode;
an instruction to transition the wireless data transmitter from a searching for a connection mode to a transmitting data mode;
an instruction to save physiological measurements to the memory store; and
an instruction to determine a specific connection polling interval.

21. A system according to claim 1 further comprising a patient interface comprising at least in part an input device.

22. A system according to claim 21 wherein the input device is a button.

23. A system according to claim 21 wherein the controller will transition the wireless data transmitter from the low power operation mode to the searching for a connection mode after the input device has been activated.

24. A system according to claim 21 wherein the user interface further comprises a user output device, such that the user interface is configured to run a neurobehavioral test.

25. A system according to claim 23 wherein the neurobehavioral test comprises one or more of: the psychomotor vigilance test, the motor praxis test, the visual object learning test, the fractal-2-back test, the conditional exclusion task, the matrix reasoning task, the line orientation test, the emotion recognition test, the balloon analog risk task, the digit symbol substitution test, the forward digit span, the reverse digit span, the serial addition and subtraction task, the go/no-go task, the word-pair memory task, the word recall test, the motor skill learning task, the threat detect test, the descending subtraction task, the PANAS-X questionnaire, the pre-sleep/post-sleep questionnaires for astronauts, the Beck depression inventory, the conflict questionnaire, the Karolinska drowsiness scales, the visual analog scales, the Karolinska sleepiness scales, the POMS/POMS-SF questionnaires, and the Stroop test.
